(19) United States
(12) Patent Application Publication
(10) Pub. No.: US 2014/0200611 A1
(43) Pub. Date: Jul. 17, 2014

(54) VASCULAR CLOSURE SYSTEM TOGGLE PLACEMENT

(71) Applicant: Greg Walters, Exton, PA (US)

(72) Inventor: Greg Walters, Exton, PA (US)

(21) Appl. No.: 14/086,418

(22) Filed: Nov. 21, 2013

Related U.S. Application Data
(60) Provisional application No. 61/752,115, filed on Jan. 14, 2013.

(51) Int. Cl. A61B 17/00

(52) U.S. Cl. CPC A61B 17/0057
USPC 606/213

(57) ABSTRACT

Methods of controlling the orientation of a toggle of a puncture sealing device are disclosed. In one example, a proximal end of a toggle of the sealing device can be trapped between a release tube and a delivery tube while the puncture sealing device is being moved into a vessel through a puncture site of the vessel. Once inserted at least one of the release tube and the delivery tube can be moved relative to the other to thereby remove the proximal end of the toggle from between the release tube and delivery tube. Subsequently at least one of the release tube and the delivery tube can be moved again relative to the other such that a distal end of the release tube abuts the toggle to thereby orient the toggle in a sealing position.

### CROSS REFERENCE TO RELATED APPLICATIONS

This application claims the benefit of U.S. Provisional Application Ser. No. 61/752,115 filed Jan. 14, 2013, the contents of which are hereby incorporated by reference as if set forth in their entirety, herein.

### BACKGROUND

Percutaneous access of the vascular system for vascular device delivery is a common medical procedure. Typically this involves using a hollow needle to puncture a vessel, then introducing an introducer sheath to open the puncture site for the introduction of catheters and wire guides for navigation through the vascular system to facilitate delivery. For example, in many cases, vascular access requires introduction of catheters and wire guides through the femoral artery. Once the procedure is completed, the devices are removed from the patient and pressure is applied to the puncture site to stop the bleeding. Thereafter, the puncture may be sealed using a closure device.

Closure devices generally consist of three basic sealing components: a toggle (or anchor) member, a sealing member (or plug), and a filament (or suture). To lock the components together within the puncture, a locking member may be used. It has been found, that during the use of such closure devices, it may be desired to maintain toggle orientation throughout the entire medical procedure.

### SUMMARY

In one embodiment, a method of sealing a puncture site in a vessel, can include the step of positioning an access sheath such that a portion of the access sheath is disposed within the vessel through the puncture site. The access sheath can define a distal end, a proximal end, and an access channel that extends from the proximal end to the distal end along an insertion direction. The method can further include the step of positioning a puncture sealing device into the access channel along the insertion direction. The sealing device can have a release tube, a delivery tube disposed within the release tube, a plug disposed within the delivery tube, a toggle distal to the plug, and a filament that couples the toggle to the plug. The toggle can define a distal end that is distal to a distal end of the release tube and a proximal end that is disposed within the release tube. The method can further include the steps of translating the sealing device within the access channel along the insertion direction such that the distal end of the toggle protrudes from a distal end of the access sheath and into the vessel, moving at least one of the delivery tube and the release tube relative to the other such that the proximal end of the toggle is released from the release tube, and again moving at least one of the delivery tube and the release tube relative to the other such that a distal end of the release tube abuts the toggle to thereby orient the toggle in a sealing position.

In another embodiment, a method of controlling the orientation of a toggle of a puncture sealing device can include the steps of, trapping a proximal end of the toggle between the release tube and the delivery tube while the puncture sealing device is being moved into a vessel through a puncture site of the vessel, moving at least one of the release tube and the delivery tube relative to the other to thereby remove the proximal end of the toggle from between the release tube and delivery tube, and again moving at least one of the release tube and the delivery tube relative to the other such that a distal end of the release tube abuts the toggle to thereby orient the toggle in a sealing position.

### BRIEF DESCRIPTION OF THE DRAWINGS

The foregoing summary, as well as the following detailed description of an example embodiment of the application, will be better understood when read in conjunction with the appended drawings, in which there is shown in the drawings example embodiments for the purposes of illustration. It should be understood, however, that the application is not limited to the precise arrangements and systems shown. In the drawings:

FIG. 1 is a perspective view of a puncture sealing device in accordance with an embodiment, the puncture sealing device being slidable along a guide wire and having a deployment device and a closure device disposed within the deployment device;

FIG. 2 is a sectional view showing a toggle of the closure device trapped between a release tube of the deployment device and a delivery tube of the deployment device;

FIG. 3A is a schematic showing an access sheath partially disposed within a vessel through a puncture site in the vessel;

FIG. 3B is a schematic showing the closure device of FIG. 1 translated into an access channel of the access sheath such that a distal end of the toggle is positioned distal to a distal end of the access sheath;

FIG. 3C is a schematic showing the access sheath and closure device combination pulled proximally such that the toggle is proximate to the puncture site;

FIG. 3D is a schematic showing the release tube being moved proximally relative to the delivery tube to thereby release the toggle;

FIG. 3E is a schematic showing the release tube being moved distally relative to the delivery tube such that the release tube abuts the toggle to thereby orient the toggle in a sealing position;

FIG. 3F is a schematic showing the deployment device being pulled proximally such that the toggle abuts the vessel wall and a plug that is coupled to the toggle with a filament is deployed from the delivery tube;

FIG. 3G is a schematic showing the plug being pressed against the vessel wall with a locking member while the guide wire remains in place;

FIG. 3H is a schematic showing the locking member being tamped against the plug with a tamper of the closure device after the guide wire has been removed; and

FIG. 3I is a schematic showing the puncture site fully sealed.

### DETAILED DESCRIPTION OF ILLUSTRATIVE EMBODIMENTS

Certain terminology is used in the following description for convenience only and is not limiting. The words “right”, “left”, “lower” and “upper” designate directions in the drawings to which reference is made. The words “proximally” and “distally” refer to directions toward and away from, respectively, the individual operating the system. The terminology includes the above-listed words, derivatives thereof and words of similar import.

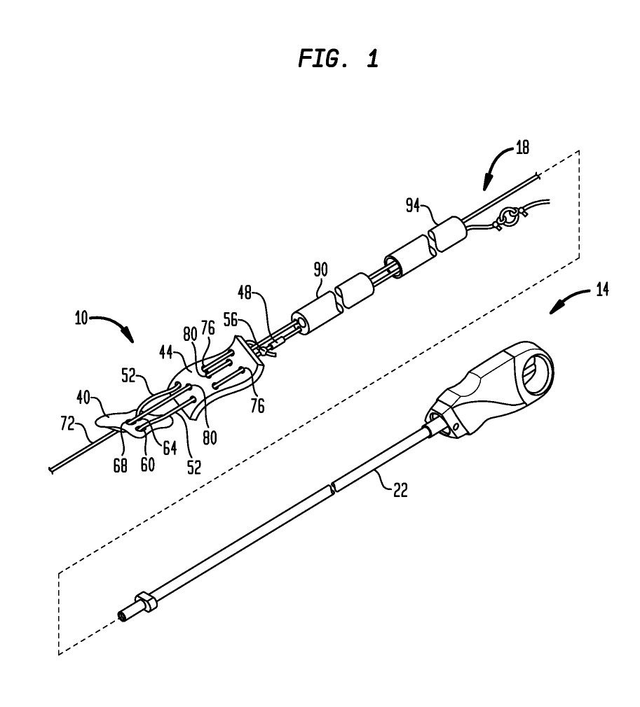

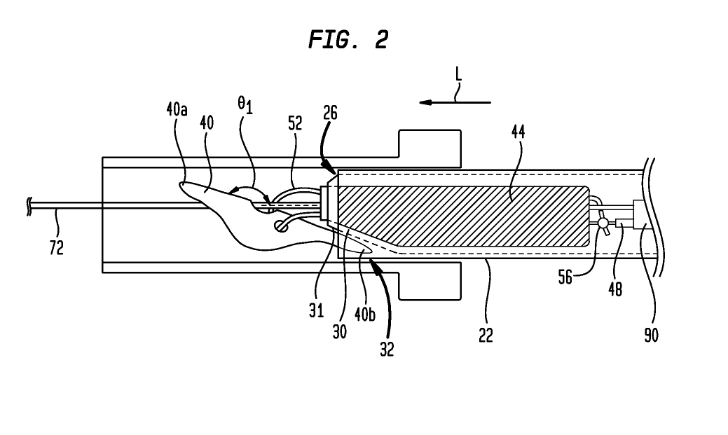

Referring to FIGS. 1 and 2 a puncture sealing device 10 in accordance with an embodiment of the invention can include a deployment device 14 and a closure device 18 at least partially disposed within the deployment device 14. After the deployment device is inserted into a vessel through a puncture site of the vessel, the closure device 18 is deployed from the deployment device 14 to thereby seal or otherwise close the puncture site of the vessel.

As shown in FIG. 2 the deployment device 14 includes a release tube 22 that is elongate along a first direction L and defines a release tube channel 26 that extends through the release tube 22 along the first direction L. The release tube 22 is configured to restrain a toggle 40 of the closure device 18 during insertion of the sealing device into the vessel and subsequently release the toggle 40 so that the toggle 40 can be oriented for the sealing procedure.

As shown in FIG. 2, the deployment device 14 further includes a delivery tube 30 that is disposed within the release tube channel 26 such that at least one of the release tube 22 and the delivery tube 30 is movable relative to the other along the first direction L. Therefore, the release tube 22 and the delivery tube 30 can be configured such that at least one of the release tube 22 and the delivery tube 30 is movable relative to the other to thereby release the toggle 40 and subsequently orient the toggle 40 for the sealing procedure.

As shown in FIG. 2, the delivery tube 30 includes an angled portion 31 at its distal end. The angled portion 31 angles toward a central axis of the delivery tube 30 such that a retention cavity 32 is defined between the angled portion 31 and the release tube 22. The retention cavity 32 is sized to receive and retain a portion of the toggle 40 to thereby trap the toggle 40 between the delivery tube 30 and the release tube 22 such that the toggle 40 is angled by a first angle Ø₁ relative to a central axis of the release tube 22. While the toggle 40 is trapped, the closure device 18 and deployment device 14 can be inserted into the vessel.

As shown in FIG. 2, the closure device 18 is at least partially disposed within the delivery tube 30 prior to being inserted into the vessel. As shown in FIGS. 1 and 2, the closure device 18 further includes a plug 44 (e.g. collagen pad), a locking member 48, and a filament 52 that couples the toggle 40, plug 44, and locking member 48 together such that the toggle 40 is distal to the plug 44 and the locking member 48 is proximal to the plug 44. As shown in FIG. 1, the filament 52 extends through the locking member 48, plug 44, and toggle 40 in the first direction L and then back through the toggle 40 and plug 44 in a direction opposite the first direction L. An end of the filament 52 is then formed into a slidable knot 56 that is slidable along the filament 52 between the plug 44 and the locking member 48. In operation, the locking member 48 and toggle 40 squeeze the plug 44 against the puncture site to thereby seal the puncture site.

The toggle 40 can be an elongate, low profile member that is configured to be seated inside the vessel against the vessel wall contiguous with the puncture site. The toggle 40 defines a distal end 40a that is distal to a distal end of the release tube 22 and a proximal end 40b that is trapped within the retention cavity 32 between the release tube 22 and the delivery tube 30 during insertion of the toggle 40 into the vessel. As shown in FIG. 1, the toggle further defines a first filament receiving aperture 60 that receives the filament 52 as it passes through the toggle 40 in the first direction L, a second filament receiving aperture 64 that receives the filament 52 as it passes through the toggle 40 in the second direction, and a guide wire aperture 68 that is configured to receive a guide wire 72 such that the closure device 18 translates along the guide wire 72 and is guided toward the puncture site by the guide wire 72. The toggle 40 can be made of any desired material. For example, the toggle 40 can made of a polylactic-coglycolic acid or other synthetic absorbable polymer that degrades in the presence of water into naturally occurring metabolites. It should be appreciated, however, that the toggle 40 can be made of other materials and can have other configurations so long as it can be seated inside the vessel against the vessel wall.

With continued reference to FIG. 1, the plug 44 is coupled to the filament 52 between the toggle 40 and the locking member 48. Like the toggle 40, the plug 44 can have a series of filament receiving apertures 76 that receive the filament 52 along the first and second directions to thereby couple the plug 44 to the filament. The plug 44 can further include a series of guide wire apertures 80 that receive the guide wire 72 during insertion of the closure device 18 into the vessel. The plug 44 can comprise a strip of compressible, resorbable, collagen foam and can be made of a fibrous collagen mix of insoluble and soluble collagen that is cross linked for strength. It should be appreciated, however, that the plug 44 can have any configuration as desired and can be made from any material as desired.

With continued reference to FIG. 1, the locking member 48 is configured to frictionally engage the filament 52 as the locking member 48 is moved along the filament 52 toward the toggle 40 to thereby seal the puncture site. That is, the locking member 48 is configured to remain in place on the filament 52 when no force is placed on the locking member 48, and only overcomes its frictional engagement with the filament 52 in response to an application of force on the locking member 48. The locking member 48 can be configured as a cylindrical member that is crimped onto the filament 52. It should be appreciated, however, that the locking member 48 can have other configurations as desired. For example, the locking member 48 can be the slideable knot 56. In such an embodiment, the slidable knot 56 can be a locking knot.

As shown in FIG. 1, the closure device 18 further includes a tamper 90 proximal to the locking member 48 and a tensioning device 94 proximal to the tamper 90. As shown, the guide wire 72 and the filament 52 extend through both the tamper 90 and the tensioning device 94. The tamper 90 is configured to be translated along the filament 52 to thereby move the locking member 48 against the plug 44. In this way, the puncture site can be fully sealed. The tensioning device 94 is configured to maintain the filament 52 in tension during the sealing procedure.

Embodiments of the present technology will now be described with respect to exemplary large bore procedures that utilize the puncture sealing device 10. In order to perform any of the related procedures, the user gains percutaneous access to, for example, the femoral artery, causing a puncture site in the artery. To gain percutaneous access to the artery, the Seldinger technique may be used. For example, a hollow bore needle is inserted into the artery. The guide wire 72 is then advanced through the hollow needle and into the femoral artery a sufficient distance to allow removal of the needle without the guide wire 72 pulling out of the vessel. Removing the needle leaves the guide wire 72 in place, with a portion of the guide wire 72 extending into the artery. The guide wire 72, extending from outside the patient into the femoral artery, provides for an entry guide for other medical devices including the puncture sealing device 10. Therefore, once the guide wire 72 is positioned in the vessel of the patient, catheters, or introducers, or gradually increasing diameters are advanced over the guidewire and through the puncture into the artery to further open the puncture site. Then, an introducer/procedure access sheath set (i.e. an introducer inside an access tube or sheath) is moved along the guide wire 72 such that a distal end of the sheath moves into the vessel through the puncture site. And once positioned, the introducer can be removed such that the sheath provides for sizable access to the vessel interior from outside the body.

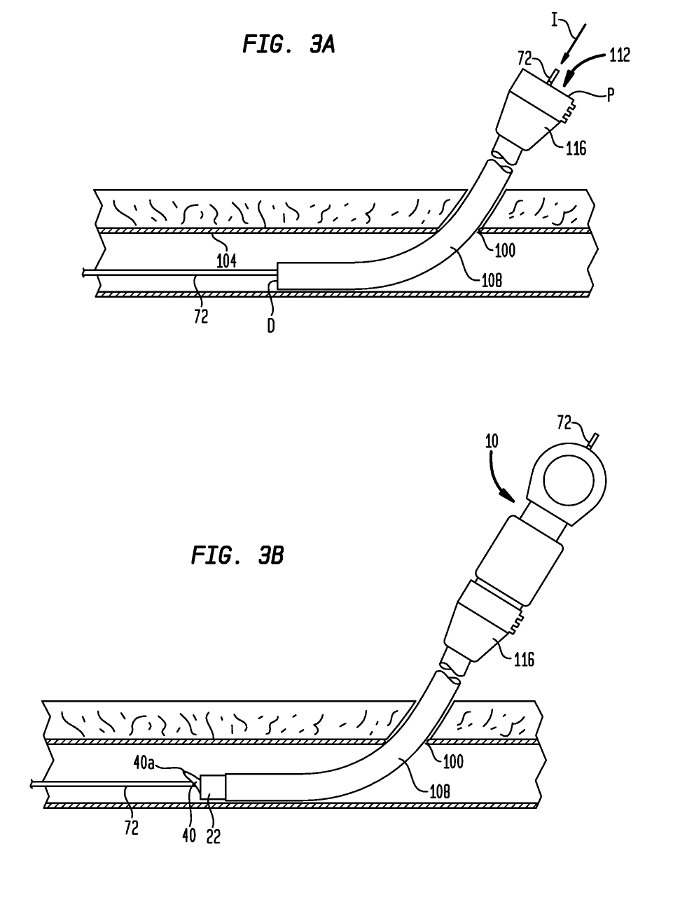

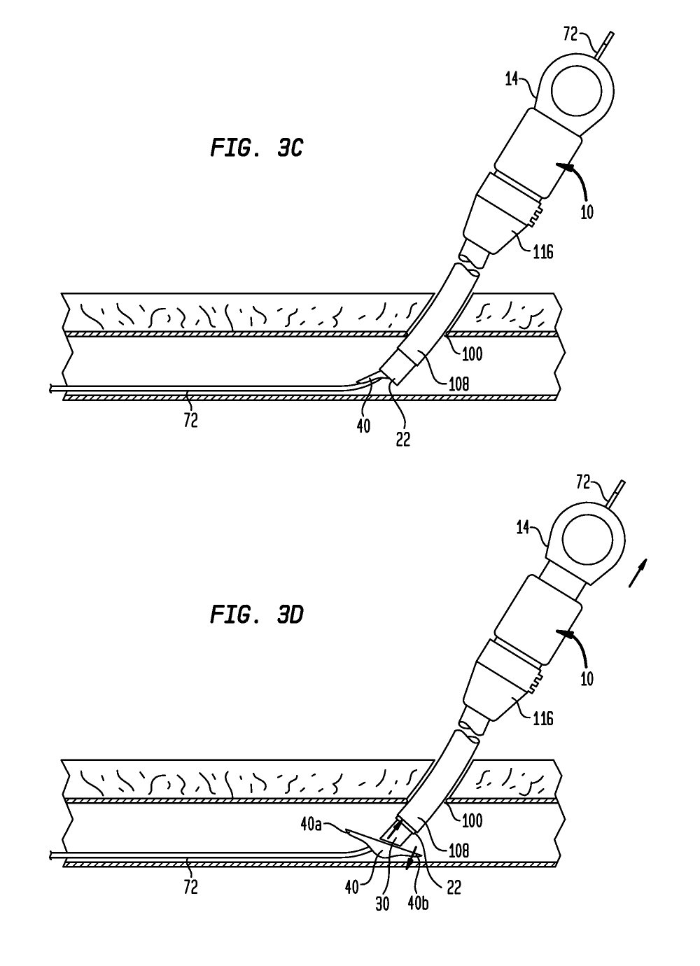

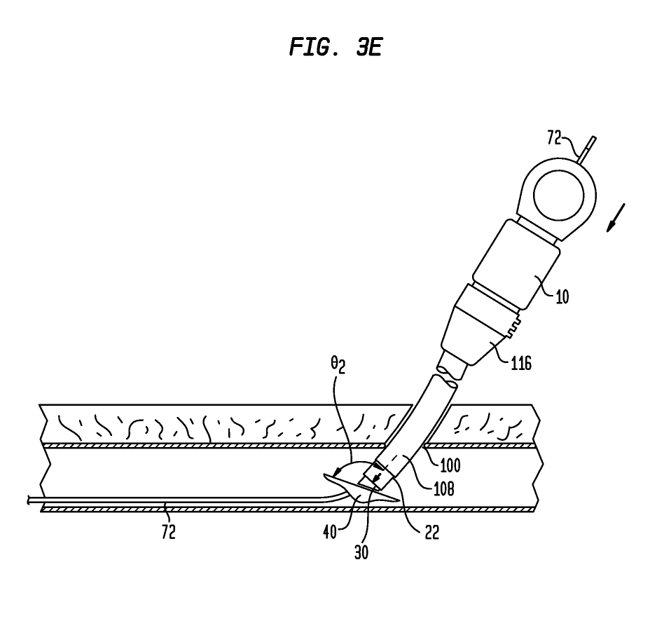

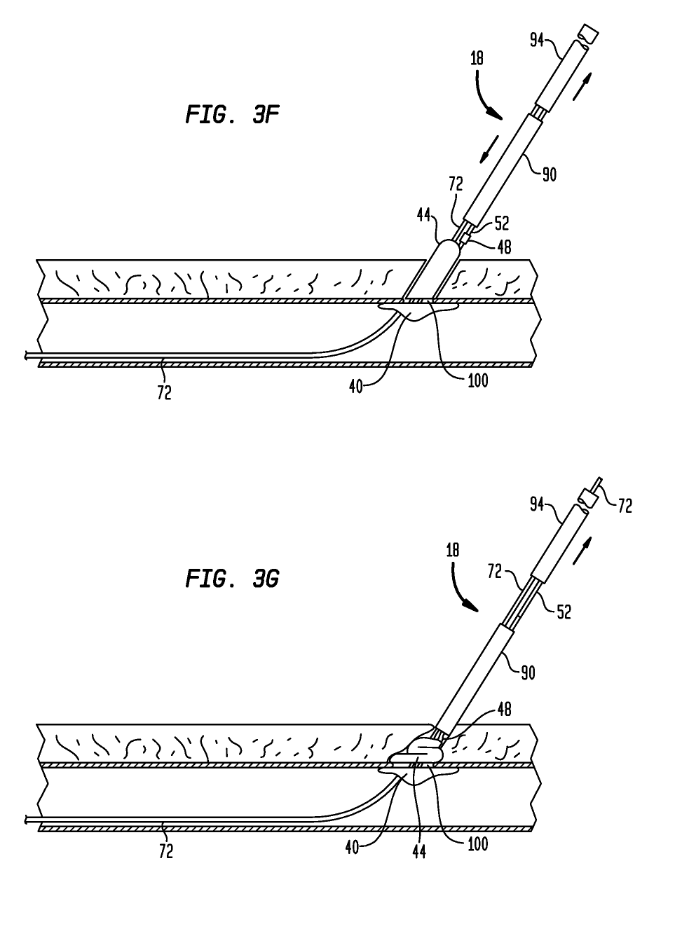

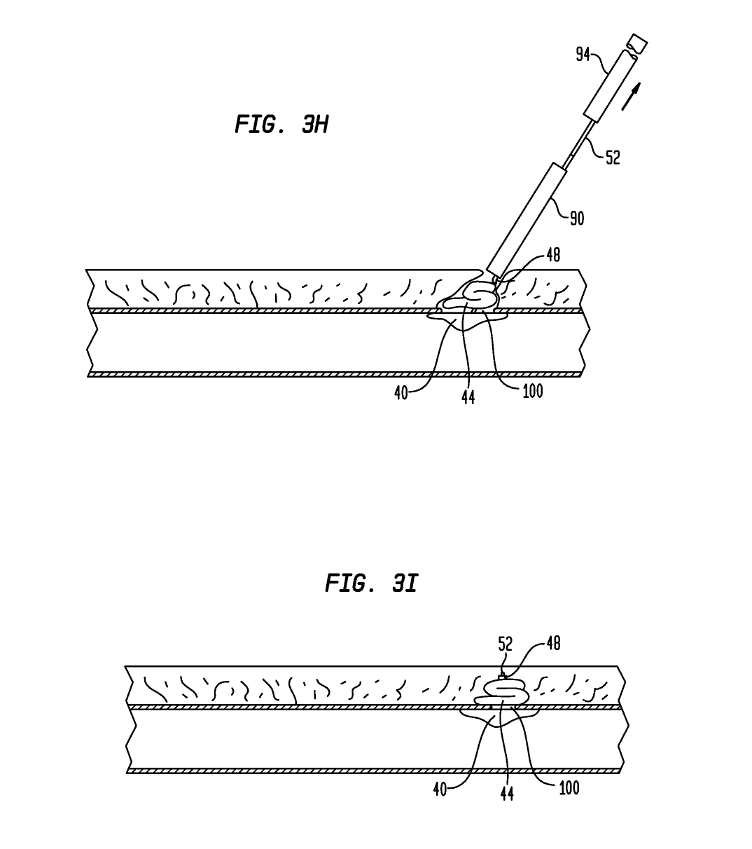

After the relevant procedure is completed, the puncture site in the artery created by the bore needle during percutaneous access of the artery may be closed. The puncture sealing device 10 may be used to seal the puncture site. FIGS. 3A-3I show schematic views of the puncture sealing device 10 during the process of closing a puncture site 100 in a vessel (e.g. artery) wall 104.

Now in reference to FIG. 3A, to deliver the puncture sealing device 10 to the puncture site 100 so that the closure device 18 can seal the puncture site 100, the introducer/procedure sheath set is replaced with a closure access sheath 108. For example, as shown in FIG. 3A, the procedure sheath is exchanged for the closure access sheath 108 by removing the procedure sheath from the patient, leaving the guide wire 72 in place, and subsequently moving the closure access sheath 108 along the guide wire 72 or otherwise positioning the access sheath 108, such that a portion of the access sheath 108 is disposed within the vessel through the puncture site 100. As shown in FIG. 3A, the access sheath 108 defines a distal end D, a proximal end P, and an access channel 112 that extends from the proximal end P to the distal end D along an insertion direction I. The access sheath 108 further includes a sheath hub 116 at its proximal end P. The sheath hub 116 is configured to couple to the puncture sealing device 10 when the puncture sealing device 10 is inserted into the access channel 112 along the insertion direction I.

As shown in FIG. 3B, the puncture sealing device 10 can be positioned by translating the puncture sealing device 10 into the access channel 112 along the insertion direction I such that at least the distal end 40a of the toggle 40 protrudes from the distal end D of the access sheath 108 and into the vessel. Once fully inserted, the puncture sealing device 10 can couple to the sheath hub 116. As shown in FIG. 3B, the proximal end 40b of the toggle 40 is trapped within the retention cavity 32 between the release tube 22 and the delivery tube 30 while the puncture sealing device 10 is being moved into the vessel through the puncture site 100 of the vessel. While the proximal end 40b of the toggle 40 is trapped, the toggle 40 is oriented in a pre-sealing position whereby at least the proximal end 40b of the toggle 40 is prevented from dragging against the vessel wall during positioning of the toggle 40 within the vessel.

Once the puncture sealing device 10 is properly positioned within the access sheath 108, the toggle 40, and in particular, the entire access sheath 108 and puncture sealing device 10 combination can be moved proximally such that the toggle 40 is adjacent the puncture site 100. While the toggle 40 is being positioned adjacent the puncture site 100 the toggle 40 is in the pre-sealing position as shown in FIG. 3C. And once the toggle 40 is in position, at least one of the delivery tube 30 and the release tube 22 can be moved relative to the other such that the proximal end 40b of the toggle 40 is released from the release tube 22 or is otherwise removed from the retention cavity 32 defined between the release tube 22 and the delivery tube 30. In the illustrated embodiment, and in reference to FIG. 3D, the release tube 22 is moved proximally relative to the delivery tube 30 to thereby release the proximal end 40b of the toggle 40 from the retention cavity 32.

As shown in FIG. 3E, at least one of the delivery tube 30 and the release tube 22 can be moved relative to the other such that a distal end of the release tube 22 abuts the toggle 40 to thereby orient the toggle 40 in a sealing position whereby the toggle is angled by a second angle Ø₂ relative to the central axis of the release tube 22 that is different than the first angle Ø₁. In particular, the second angle Ø₂ is smaller than the first angle Ø₁.

In the illustrated embodiment, the release tube 22 is moved distally relative to the delivery tube 30 so that the release tube 22 can abut the toggle 40 and orient it in the sealing position. As shown in FIG. 3E, the toggle 40 is angled relative to the delivery tube 30 when in the sealing position. The angled orientation of the toggle 40 is such that the toggle 40 remains within the vessel when the toggle 40 is pulled against the vessel wall 104.

While the toggle 40 is in the sealing position, a tension can be applied to the filament 52. For example, the filament 52 can be pulled proximally relative to the delivery tube 30 to thereby ensure that the toggle 40 remains in the sealing position whereby the toggle 40 abuts the release tube 22. The tension can be applied to the filament prior to the release tube 22 being moved to abut the toggle 40, after the release tube 22 has been moved to abut the toggle 40, or at the same time the release tube 22 is being moved.

With the toggle 40 in the sealing position as shown in FIG. 3E, the delivery device 14 along with the access sheath 108 can together be pulled proximally such that the plug 44 and other components of the closure device 18 emerge from the delivery tube 30. As shown in FIG. 3F, the closure device 18, including the toggle 40, plug 44, locking member 48, filament 52, tamper 90, and tensioning device 94, are fully withdrawn from the delivery tube 30. By pulling on the tensioning device 94 in a direction away from the vessel (i.e. in a direction opposite the insertion direction I) the filament 52 is tensioned and the toggle 40 is moved fully into position against an inner surface of the vessel wall 104 at the puncture site 100. The tension in the filament 52 also pulls the plug 44 into the puncture site 100, and causes the plug 44 to substantially fill the puncture site 100 as shown in FIG. 3F. After the plug 44 is in contact with blood or other fluids within the puncture site 100, the plug 44 will expand and fill the remainder of the puncture site 100.

After the user has pulled the tensioning device 94 to cause tension in the filament 52 and to cause the plug 44 to enter the puncture site 100, the user advances the tamper 90 along the guide wire 72 and the filament 52. As shown in FIG. 3G, the tamper 90 contacts the locking member 44 and advances the locking member 44 along the filament 52 until the locking member 44 contacts the plug 44 and presses the plug 44 against an outer surface of the vessel. As the plug 44 is compressed by the tamper 90 the plug 44 folds over the top of and inside the puncture site 100. It should be appreciated, however, that in some embodiments, the delivery tube 30 is pulled such that the plug 44 is removed from the delivery tube 30 within the release tube 22 and the tamper 90 is employed within the release tube 22. In such an embodiment, the release tube 22 helps control the plug 44 as it is being tamped against the puncture site.

As shown in FIGS. 3H and 3I, the locking member 44, together with the plug 44 and the toggle 40 effect a seal of the puncture site 100. As shown in FIG. 3H, tension is maintained on the tensioning device 94 throughout the deployment of the plug 44 from the delivery tube 30. After the puncture site 100 is sealed, the guide wire 72 can be removed as shown in FIG. 3H. As the guide wire 72 is removed, the filament 52 remains in tension and the user can re-compress the plug 44 with the tamper 90 as desired to confirm a proper seal of the puncture site 100. Once properly sealed, the filament 52 can be cut below the tamper 90 so that the remaining filament 52, tamper 90, and tensioning device 94 can be removed from the puncture site 100, as shown in FIG. 3I. Remaining portions of the closure device 18, including the toggle 40, plug 44, portion of filament 52, and locking member 48 (depending on material used) will resorb into the body of the patient over time.

While the foregoing description and drawings represent the preferred embodiment of the present invention, it will be understood that various additions, modifications, combinations and/or substitutions may be made therein without departing from the spirit and scope of the invention as defined in the accompanying claims. In particular, it will be clear to those skilled in the art that the invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components, which are particularly adapted to specific environments and operative requirements without departing from the principles of the invention. In addition, features described herein may be used singularly or in combination with other features. For example, features described in connection with one component may be used and/or interchanged with features described in another component. The presently disclosed embodiment is therefore to be considered in all respects as illustrative and not restrictive, the scope of the invention being indicated by the appended claims, and not limited to the foregoing description.

It will be appreciated by those skilled in the art that various modifications and alterations of the invention can be made without departing from the broad scope of the appended claims. Some of these have been discussed above and others will be apparent to those skilled in the art.

What is claimed:

1. A method of sealing a puncture site in a vessel, the method comprising the steps of:
positioning an access sheath such that a portion of the access sheath is disposed within the vessel through the puncture site, the access sheath defining a distal end, a proximal end, and an access channel that extends from the proximal end to the distal end along an insertion direction;
positioning a puncture sealing device into the access channel along the insertion direction, the sealing device having a release tube, a delivery tube disposed within the release tube, a plug disposed within the delivery tube, a toggle distal to the plug, and a filament that couples the toggle to the plug, the toggle defining at least a proximal end that is disposed within the release tube;
translating the sealing device within the access channel along the insertion direction such that the toggle protrudes from a distal end of the access sheath and into the vessel;
moving at least one of the delivery tube and the release tube relative to the other such that the proximal end of the toggle is released from the release tube; and
again moving at least one of the delivery tube and the release tube relative to the other such that a distal end of the release tube abuts the toggle to thereby orient the toggle in a sealing position.

2. The method of claim 1, wherein the first moving step comprises moving the release tube proximally relative to the delivery tube.

3. The method of claim 2, wherein the second moving step comprises moving the release tube distally relative to the delivery tube.

4. The method of claim 1, further comprising moving the toggle proximally such that the toggle is adjacent the puncture site while the toggle is in the sealing position.

5. The method of claim 4, further comprising pulling at least the delivery tube proximally such that the plug is removed from the delivery tube.

6. The method of claim 5, further comprising the step of tamping the plug against the puncture site to thereby seal the puncture site.

7. The method of claim 6, wherein the pulling step comprises pulling the delivery tube proximally such that the plug is removed from the delivery tube within the release tube and the tamping step is performed within the release tube.

8. The method of claim 1, further comprising the step of providing tension to the filament while the toggle is in the sealing position.

9. The method of claim 8, wherein the tension is provided prior to the second moving step.

10. A method of controlling the orientation of a toggle of a puncture sealing device, the puncture sealing device further having a release tube, a delivery tube disposed within the release tube, and a plug disposed within the delivery tube and coupled to the toggle, the method comprising the steps of:
trapping a proximal end of the toggle between the release tube and the delivery tube while the puncture sealing device is being moved into a vessel through a puncture site of the vessel;
moving at least one of the release tube and the delivery tube relative to the other to thereby remove the proximal end of the toggle from between the release tube and delivery tube; and
again moving at least one of the release tube and the delivery tube relative to the other such that a distal end of the release tube abuts the toggle to thereby orient the toggle in a sealing position.

11. The method of claim 10, further comprising the step of applying tension to a filament that is coupled to the toggle while the toggle is in the sealing position.

12. The method of claim 11, wherein the tension is applied prior to the second moving step.

13. The method of claim 11, wherein the puncture sealing device further includes a filament that couples the plug to the toggle, and wherein the applying step comprises pulling the filament proximally relative to the delivery tube.

14. The method of claim 10, wherein the toggle is angled relative to the delivery tube when in the sealing position.

15. The method of claim 10, wherein the first moving step comprises moving the release tube proximally relative to the delivery tube.

16. The method of claim 15, wherein the second moving step comprises moving the release tube distally relative to the delivery tube.

17. A method of sealing a puncture site in a vessel, the method comprising the steps of:
positioning an access sheath such that a portion of the access sheath is disposed within the vessel through the puncture site, the access sheath defining a distal end, a proximal end, and an access channel that extends from the proximal end to the distal end along an insertion direction;
positioning a puncture sealing device into the access channel along the insertion direction, the sealing device having a release tube, a delivery tube disposed within the release tube, a plug disposed within the delivery tube, a toggle distal to the plug, and a filament that couples the toggle to the plug, at least a portion of the toggle being restrained by at least one of the release tube and the delivery tube, such that the toggle is angled by a first angle relative to a central axis of the release tube;
while the toggle is restrained, translating the sealing device within the access channel along the insertion direction such that the toggle protrudes from a distal end of the access sheath and into the vessel;
releasing the toggle such that the toggle is no longer restrained by the release tube and the delivery tube; and
orienting the toggle into a sealing position with the release tube, such that the toggle is angled by a second angle relative to the central axis that is different than the first angle.

18. The method of claim 17, wherein the toggle is restrained by trapping a proximal end of the toggle between the release tube and the delivery tube.

19. The method of claim 18, wherein the releasing step comprises moving at least one of the delivery tube and the release tube relative to the other such that the proximal end of the toggle is released from the release tube.

20. The method of claim 19, wherein the releasing step comprises moving the release tube proximally relative to the delivery tube.

21. The method of claim 19, wherein the orienting step comprises, moving at least one of the delivery tube and the release tube relative to the other such that a distal end of the release tube abuts the toggle to thereby orient the toggle in the sealing position.

22. The method of claim 21, wherein the orienting step comprises moving the release tube distally relative to the delivery tube.

23. The method of claim 18, further comprising the step of providing a tension to the filament while the toggle is in the sealing position.

24. The method of claim 23, wherein the tension is provided prior to the orienting step.

25. The method of claim 17, wherein the first angle is greater than the second angle.
